(19) United States
(12) Patent Application Publication
(10) Pub. No.: US 2022/0386908 A1
(43) Pub. Date: Dec. 8, 2022

(54) DEVICE AND METHOD OF DETECTING AND CALIBRATING A VOLTAMMETRIC RESPONSE TO IN VIVO BIOCHEMICALS

(71) Applicant: THE REGENTS OF THE UNIVERSITY OF CALIFORNIA, Oakland, CA (US)

(72) Inventors: Sam Emaminejad, Los Angeles, CA (US); Shuyu Lin, Los Angeles, CA (US); Bo Wang, Los Angeles, CA (US)

(73) Assignee: THE REGENTS OF THE UNIVERSITY OF CALIFORNIA, Oakland, CA (US)

(21) Appl. No.: 17/771,440

(22) PCT Filed: Oct. 26, 2020
(86) PCT No.: PCT/US2020/057366

§ 371 Date: Apr. 22, 2022

Related U.S. Application Data
(60) Provisional application No. 62/926,100, filed on Oct. 25, 2019.

(51) Int. Cl. A61B 5/145; A61B 5/1477; A61B 5/1495; G01N 33/543

(52) U.S. Cl. CPC A61B 5/14546; A61B 5/14517; A61B 5/1477; A61B 5/1495; G01N 33/5438

(57) ABSTRACT

Example implementations include a method of applying a voltage pulse having a magnitude within a biochemical voltage window associated a biochemical, obtaining a response current from a biochemical sensor electrode, generating a biochemical response voltammogram based on the response current, extracting a current peak from the biochemical response voltammogram, and generating a biochemical concentration based on the current peak. Example implementations further include a method of applying a differential pulse sequence including the voltage pulse to the reference electrode. Example implementations further include a method of applying the differential pulse sequence further comprises applying the differential pulse sequence to the reference electrode at an increasing voltage step.

### STATEMENT REGARDING FEDERALLY SPONSORED RESEARCH OR DEVELOPMENT

This invention was made with government support under Grant Number 1847729, awarded by the National Science Foundation. The government has certain rights in the invention.

### CROSS-REFERENCE TO RELATED PATENT APPLICATIONS

This application claims priority to U.S. Provisional Patent Application Ser. No. 62/926,100, entitled “Wearable Voltammetric Monitoring of Electroactive Drugs,” filed Oct. 25, 2019, the contents of such application being hereby incorporated by reference in its entirety and for all purposes as if completely and fully set forth herein.

### TECHNICAL FIELD

The present implementations relate generally to biochemical sensors, and more particularly to detecting and calibrating a voltammetric response to in vivo biochemicals.

### BACKGROUND

Health monitoring is increasingly desired to perform increasingly accurate health diagnostics and guide improved health outcomes for increasing numbers of users and activity scenarios. In particular, detection of biochemical levels of biofluids secreted by a user can provide significant health data and, in turn, drive significantly improved health outcomes. However, conventional systems may not effectively detect and isolate biochemicals in biofluids at in vivo sites noninvasively and accurately. In addition, conventional systems may inaccurately detect amounts of one or more biofluids in the presence of one or more interferents. Thus, a technological solution for detecting and calibrating a voltammetric response to in vivo biochemicals is desired.

### SUMMARY

Example implementations include a method of applying a voltage pulse having a magnitude within a biochemical voltage window associated a biochemical, obtaining a response current from a biochemical sensor electrode, generating a biochemical response voltammogram based on the response current, extracting a current peak from the biochemical response voltammogram, and generating a biochemical concentration based on the current peak. Example implementations further include a method of applying a differential pulse sequence including the voltage pulse to the reference electrode. Example implementations further include a method of applying the differential pulse sequence further comprises applying the differential pulse sequence to the reference electrode at an increasing voltage step.

Example implementations include a device with an iontophoresis inducer configured to apply a voltage pulse to a biofluid including a biochemical, the voltage pulse having a magnitude within a biochemical voltage window associated a biochemical, a biochemical sensor electrode operatively configured to obtain a response current from the biofluid, a transimpedance amplifier operatively coupled to the biochemical sensor electrode, and configured to obtain the response current from the biochemical sensor electrode, and a system processor operatively coupled to the iontophoresis inducer and the transimpedance amplifier, and configured to generate a biochemical response voltammogram based on the response current, extract a current peak from the biochemical response voltammogram, and generate a biochemical concentration based on the current peak.

### BRIEF DESCRIPTION OF THE DRAWINGS

These and other aspects and features of the present implementations will become apparent to those ordinarily skilled in the art upon review of the following description of specific implementations in conjunction with the accompanying figures, wherein:

FIG. 1 illustrates an example biochemical sensor device, in accordance with present implementations.

FIG. 2 illustrates an example biochemical sensor, in accordance with present implementations.

FIG. 3 illustrates an example electronic sensor device, in accordance with present implementations.

FIG. 4 illustrates an example biochemical sensor response, in accordance with present implementations.

FIG. 5 illustrates an example biochemical sensor response including multiple biochemical sensor windows, in accordance with present implementations.

FIG. 6 illustrates an example biochemical sensor response including multiple interferent windows, in accordance with present implementations.

FIG. 7 illustrates an example method of electrically sensing a biochemical in accordance with present implementations.

FIG. 8 illustrates an example method of electrically sensing a biochemical, further to the example method of FIG. 7.

FIG. 9 illustrates a further example method of electrically sensing a biochemical.

### DETAILED DESCRIPTION

The present implementations will now be described in detail with reference to the drawings, which are provided as illustrative examples of the implementations so as to enable those skilled in the art to practice the implementations and alternatives apparent to those skilled in the art. Notably, the figures and examples below are not meant to limit the scope of the present implementations to a single implementation, but other implementations are possible by way of interchange of some or all of the described or illustrated elements. Moreover, where certain elements of the present implementations can be partially or fully implemented using known components, only those portions of such known components that are necessary for an understanding of the present implementations will be described, and detailed descriptions of other portions of such known components will be omitted so as not to obscure the present implementations. Implementations described as being implemented in software should not be limited thereto, but can include implementations implemented in hardware, or combinations of software and hardware, and vice-versa, as will be apparent to those skilled in the art, unless otherwise specified herein. In the present specification, an implementation showing a singular component should not be considered limiting; rather, the present disclosure is intended to encompass other implementations including a plurality of the same component, and vice-versa, unless explicitly stated otherwise herein. Moreover, applicants do not intend for any term in the specification or claims to be ascribed an uncommon or special meaning unless explicitly set forth as such. Further, the present implementations encompass present and future known equivalents to the known components referred to herein by way of illustration.

Wearable drug monitoring targeting epidermally-retrievable biofluids (e.g., sweat) can enable a variety of applications, including drug compliance/abuse monitoring and personalized therapeutic drug dosing. In some implementations, voltammetry-based approaches uniquely leverage the electroactive nature of target drug molecules for quantification, eliminating the reliance on the availability of recognition elements. In some implementations, example implementations advantageously address one or more challenges, including constructing a sensitive voltammetric sensing interface with high signal-to-background ratio, decoupling the confounding effect of endogenous electroactive species, mitigating baseline input variation, and realizing wireless voltammetric excitation and signal acquisition/transmission. In some implementations, one or more of various endogenous electroactive species and baseline inputs are naturally present in complex biofluid matrix.

In some implementations, sweat analysis non-invasively provides proxy measures of target drug concentration in blood. Thus, sweat analysis is advantageous to therapy management, including drug compliance/abuse monitoring, drug-drug interaction study, and personalized dosing. Because of the exogenous nature of various biochemicals, natural biological recognition elements for drug molecules are usually not available. As one example, biological recognition elements include enzymes and antibodies. Given their low concentration in biofluids, it is advantageous to provide a technological solution for selective and sensitive measurement in the presence of highly abundant non-target/interfering species.

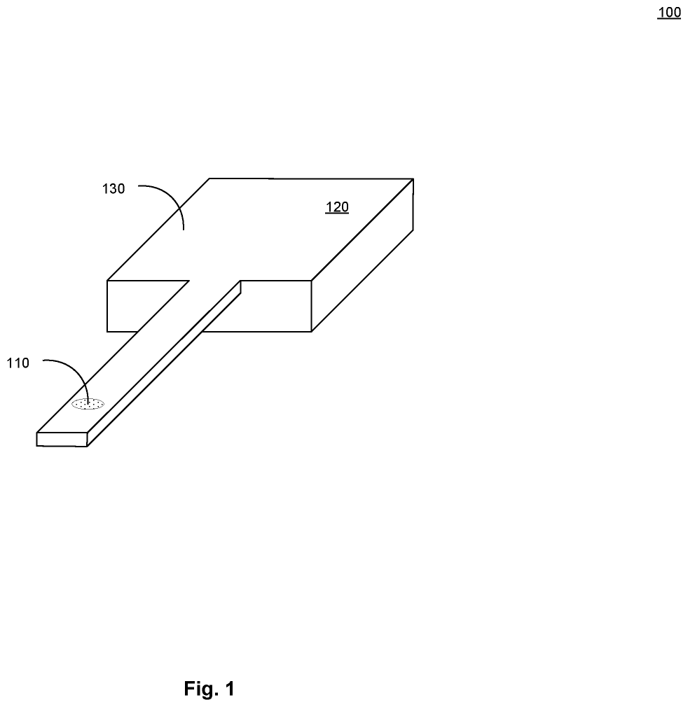

FIG. 1 illustrates an example biochemical sensor device, in accordance with present implementations. As illustrated by way of example in FIG. 1, an example biochemical sensor device 100 includes a biochemical sensor surface 110, a housing 120, and an electronics portion 130 of the sensor device housing 120.

The biochemical sensor surface 110 is operable to contact with, provide electrical stimulation to, and receive an electrochemical response from, a biological surface. In some implementations, the biochemical sensor surface is a substrate on which one or more electrical sensors, electrochemical sensors, or the like, are disposed, patterned, affixed, or the like. In some implementations, the biochemical sensor surface 110 is disposed on a flexible substrate. In some implementations, the biochemical sensor surface is a flexible solid material. In some implementations, the biochemical sensor surface is electrically coupled to one or more electrical components housed within or associated with the electronics portion 130 of the housing 120.

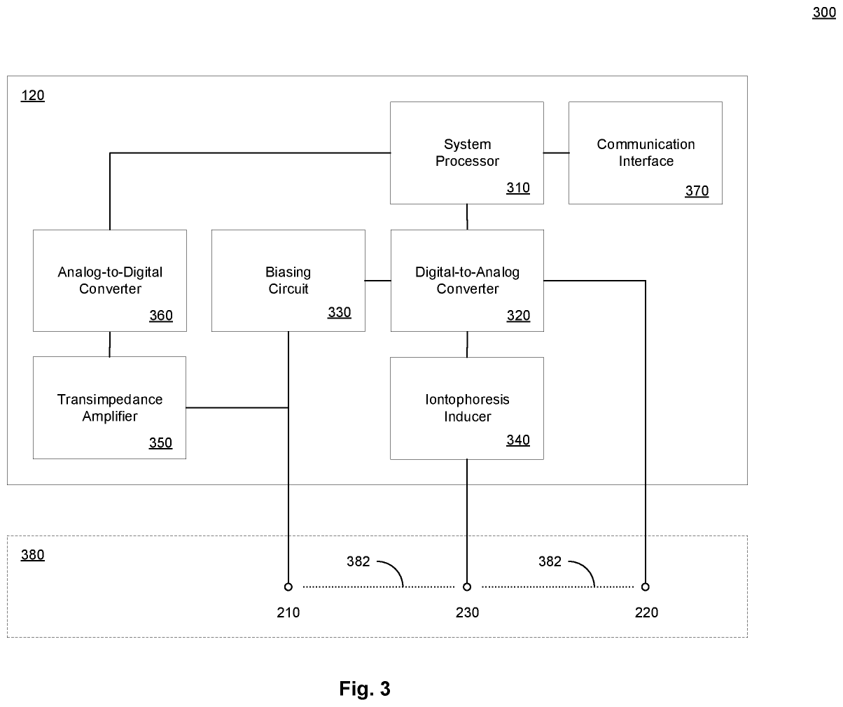

The housing 120 contains or the like one or more sensors, electrical devices, electronic devices, mechanical structures, and the like. In some implementations, the housing 120 includes a plastic material, a polymer material, electrically insulating material, waterproof material, water resistant material, or the like. The electronics portion 130 of the housing 120 houses one or more electronic components. In some implementations, the electronics portion 130 houses at least one component of FIG. 3. It is to be understood that the housing 120 and the electronics portion 130 thereof are not limited to the absolute or relative configuration of FIG. 1 in accordance with present implementations.

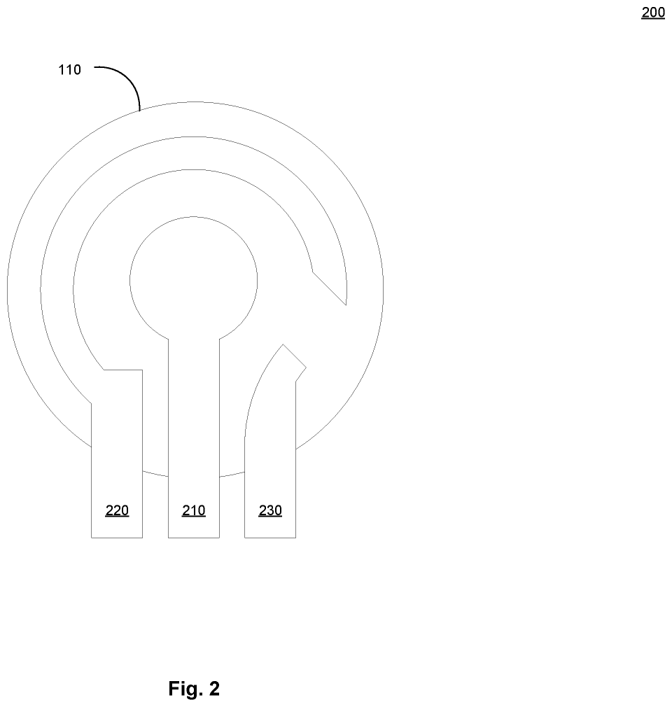

FIG. 2 illustrates an example biochemical sensor, in accordance with present implementations. As illustrated by way of example in FIG. 2, an example biochemical sensor 200 includes the biochemical sensor surface 110, a biochemical sensor electrode 210, a counter electrode 220, and a reference electrode 230. To adapt voltammetry for drug monitoring applications, a sensitive and inherently stable sensing electrode need to be selected.

The biochemical sensor electrode 210 is operable to receive a response current responsive to the presence of a biochemical. In some implementations, the biochemical sensor electrode is a boron-doped diamond electrode (BDDE). In some implementations, BDDE possess advantageous properties including but not limited to a wide electrochemical potential window and high operational stability. In some implementations, a surface of the biochemical sensor electrode contactable with a biological surface is treated with a coating, additive, or the like. In some implementations, a biochemical sensor electrode 210 is operable to detect the presence of one or more biochemicals in a biofluid at nanomolar and micromolar levels. Because of its unique sp3 diamond structure, BDDE advantageously manifests various electrochemical sensing properties including a wide electrochemical potential window, low background current, high fouling resistance, high biocompatibility, relatively rapid electron transfer kinetics, and long term stability under high-potential operation.

In some implementations, the biochemical sensor electrode 210 is an anodic-treated BDDE. In some implementations, a potential of +2 V vs. silver/silver chloride is applied to the BBDE for 5 min in 0.5 M sulfuric acid. In some implementations, BDDE manifest a double layer capacitance of substantially 8 μF/cm2, indicating a low background current when applied in voltammetric measurements.

The counter electrode 220 is optionally integrated into the example biochemical sensor 200. In some implementations, the counter electrode is a glassy carbon electrode (GCE). In some implementations, a GCE is pretreated by polishing with diamond suspension (1 μm) and ultrasonicated in ethanol for 5 min and deionized (DI) water for 5 min. In some implementations, the counter electrode 220 is a screen printed carbon electrode. The reference electrode 230 is operable to apply one or more current pulses to a biological surface. In some implementations, the reference electrode 230 is or includes silver chloride.

FIG. 3 illustrates an example electronic sensor device, in accordance with present implementations. As illustrated by way of example in FIG. 3, example electronic sensor device 300 includes the electronics region 130 of the sensor device housing 120. In some implementations, the electronics region 130 includes the biochemical sensor electrode 210, the counter electrode 220, the reference electrode 230, a system processor 310, a digital-to-analog converter (DAC) 320, a biasing circuit 330, an iontophoresis inducer 340, a transimpedance amplifier (TIA) 350, an analog-to-digital converter (ADC) 360, and a communication interface 370. In some implementations, the example electronic sensor device 300 includes and is contactable with a biological surface 380 by one or more of the biochemical sensor electrode 210, the counter electrode 220, and the reference electrode 230. In some implementations, the example electronic sensor device interfaces with the biological surface 380 by at least one biological conductive path 382 at the biological surface 380. In some implementations, the conductive path 380 is disposed through one or more of the biochemical sensor electrode 210, the counter electrode 220, and the reference electrode 230.

The system processor 310 is operable to execute one or more instructions associated with input from at least one of the biochemical sensor surface 110 and the biochemical sensor electrode 210. In some implementations, the system processor 310 is an electronic processor, an integrated circuit, or the like including one or more of digital logic, analog logic, digital sensors, analog sensors, communication buses, volatile memory, nonvolatile memory, and the like. In some implementations, the system processor 310 includes but is not limited to, at least one microcontroller unit (MCU), microprocessor unit (MPU), central processing unit (CPU), graphics processing unit (GPU), physics processing unit (PPU), embedded controller (EC), or the like. In some implementations, the system processor 310 includes a memory operable to store or storing one or more instructions for operating components of the system processor 310 and operating components operably coupled to the system processor 310. In some implementations, the one or more instructions include at least one of firmware, software, hardware, operating systems, embedded operating systems, and the like. It is to be understood that the system processor 310 or the device 300 generally can include at least one communication bus controller to effect communication between the system processor 310 and the other elements of the device 300. In some implementations, the system processor 310 is operable to generate one or more square wave voltage pulse signal instructions to apply one or more stimulation current pulses to a biological surface. In some implementations, the system processor 310 is operable to apply the current pulses to the reference electrode directly. Alternatively, in some implementations, the system processor is operable to apply the current pulses indirectly by at least one intervening structure. In some implementations, the intervening structure is or includes the DAC 320.

The DAC 320 is operable to receive one or more digital instructions from the system processor 310 and to output one or more analog signals corresponding to the digital instructions. In some implementations, the DAC 320 is operatively coupled to the iontophoresis inducer 340 by at least one communication line, bus, or the like. In some implementations, the DAC 320 supplies one or more analog instructions to the iontophoresis inducer 340 to apply at least one current pulse, sequence of current pulses, and the like, to the reference electrode. In some implementations, the DAC 320 includes one or more logical or electronic devices including but not limited to integrated circuits, logic gates, flip flops, gate arrays, programmable gate arrays, and the like. It is to be understood that any electrical, electronic, or like devices, or components associated with the DAC 320 can also be associated with, integrated with, integrable with, replaced by, supplemented by, complemented by, or the like, the system processor 310 or any component thereof.

The biasing circuit 330 is operable to receive one or more instructions from the DAC 320 to apply an electrical bias to the biochemical sensor electrode 310. In some implementations, the biasing circuit 330 applies a constant voltage at a minimum bias voltage to the biochemical sensor electrode 230. As one example, a minimum bias voltage is equal to an activation voltage of a BDDE. In some implementations, the biasing circuit 330 includes one or more logical or electronic devices including but not limited to integrated circuits, logic gates, flip flops, gate arrays, programmable gate arrays, and the like. It is to be understood that any electrical, electronic, or like devices, or components associated with the biasing circuit 330 can also be associated with, integrated with, integrable with, replaced by, supplemented by, complemented by, or the like, the system processor 310 or any component thereof.

The iontophoresis inducer 340 is operable to control, generate, define, or the like, one or more signals, pulses, or the like, of electrical energy applied to the biological surface according to one or more electrical output patterns. In some implementations, the iontophoresis inducer 340 is operable to apply electrical energy to the biological surface in accordance with an iontophoresis process. In some implementations, the electronics portion 130 of the housing 120 includes the iontophoresis inducer 340. In some implementations, the iontophoresis inducer 340 is operable to induce a biological reaction from the biological surface in accordance with the operation of the reference electrode 230. In some implementations, the iontophoresis inducer 340 includes one or more electrical, electronic, and logical devices. In some implementations, the iontophoresis inducer 340 includes one or more integrated circuits, transistors, transistor arrays, or the like. The reference electrode 230 is operable to apply one or more signals, pulses, or the like, of electrical energy to the biological surface according to one or more electrical output patterns in response to signals, instructions, or the like received from at least one of the DAC 330 and the iontophoresis inducer 340.

In some implementations, the iontophoresis inducer 340 applies a constant voltage at a minimum stimulation voltage to the reference electrode 230. As one example, a minimum stimulation voltage is equal to a lowest voltage magnitude associated with a particular voltage window. In some implementations, the iontophoresis inducer 340 is operable to increase a stimulation voltage in accordance with a step voltage or the like. In some implementations, the iontophoresis inducer 340 is operable to increase a stimulation voltage from the minimum stimulation voltage to a maximum stimulation voltage according to the step voltage, a timing parameter, and the like. As one example, a maximum stimulation voltage is equal to a highest voltage magnitude associated with a particular voltage window. In some implementations, the iontophoresis inducer 340 is operable to apply one or more current pulses to increase or decrease the magnitude of the stimulation voltage applied by the iontophoresis inducer 340. The TIA 350 is operable to receive a response current from the biochemical sensor electrode 210. In some implementations, the biochemical sensor electrode 210 is operable to transmit a response current of varying magnitudes proportional to of one or more biochemicals present in contact therewith. In some implementations, the TIA 350 receives one or more electrical impulses at one or more current response levels, and converts the current response to a voltage response. In some implementations, the TIA 350 converts the current response to a voltage response based on an actual or estimated resistance, impedance, or like of at least one of the biological surface 380 and the biological conductive path 382. In some implementations, the TUA 350 is operable to temporarily store one or more current responses and voltage responses, at a memory device integrable, coupleable, or integrated therewith, or operably coupled thereto. In some implementations, the memory device is or includes an electrically erasable programmable read-only memory (EEPROM). In some implementations, the TIA 350 includes one or more logical or electronic devices including but not limited to integrated circuits, logic gates, flip flops, gate arrays, programmable gate arrays, and the like. It is to be understood that any electrical, electronic, or like devices, or components associated with the TIA 350 can also be associated with, integrated with, integrable with, replaced by, supplemented by, complemented by, or the like, the system processor 310 or any component thereof.

The ADC 360 is operable to receive one or more digital instructions from the TIA 350 and to output one or more analog signals corresponding to the digital instructions to the system processor 310. In some implementations, the ADC 360 is operatively coupled to the system processor 310 and the TIA 350 by at least one communication line, bus, or the like. In some implementations, the ADC 360 receives one or more analog instructions from the TIA 350 including at least one voltage response based on at least one current pulse, sequence of current pulses, and the like, from the biochemical sensor electrode 210. In some implementations, the ADC 360 includes one or more logical or electronic devices including but not limited to integrated circuits, logic gates, flip flops, gate arrays, programmable gate arrays, and the like. In some implementations, the ADC includes a 24-bit address space. It is to be understood that any electrical, electronic, or like devices, or components associated with the ADC 360 can also be associated with, integrated with, integrable with, replaced by, supplemented by, complemented by, or the like, the system processor 310 or any component thereof.

The communication interface 370 is operable to communicatively couple at least the system processor 310 to at least one external device. In some implementations, the communication interface 370 includes one or more wired interface devices, channels, and the like. In some implementations, the communication interface 370 includes, is operably coupled to, or is operably couplable to an I2C, UART, or like communication interface by one or more external devices, systems, or the like. In some implementations, the communication interface 370 includes a network or an Internet communication interface or is operably couplable to an Internet communication interface by one or more external devices, systems, or the like. In some implementations, the communication interface 370 is or includes a wireless transceiver operable to wirelessly and bilaterally communicate user commands and the sensor output current. In some implementations, the communication interface 370 is or includes a Bluetooth TM transceiver. In some implementations, the communication interface 370 communication in real-time with an external device. In some implementations, an external device includes a custom-developed computer, smartphone, tablet, or like application compatible with the output of the system processor 310.

The biological surface 380 is or includes a surface of living tissue, biological matter, or the like. In some implementations, the biological surface 380 includes partially or fully exposed skin or the like of a human, animal, plant, or the like. In some implementations, the biological surface secretes or is capable of secreting one or more fluids having one or more biochemicals therein. In some implementations, biochemicals include, but are not limited to, dipyridamole, acetaminophen, caffeine, and the like. In some implementations, a biological surface is a wrist, forearm, or the like. In some implementations, sweat is collected from at least one wrist, forearm, and the like. In some implementations, it is advantageous to obtain sweat from fingertips due to relatively high density of eccrine sweat glands therein and blood capillaries in close proximity to fingertip sweat glands. In some implementations, biological surfaces can include on-body skin sites at any location thereon, and are not limited to finger, fingertip, or like surfaces.

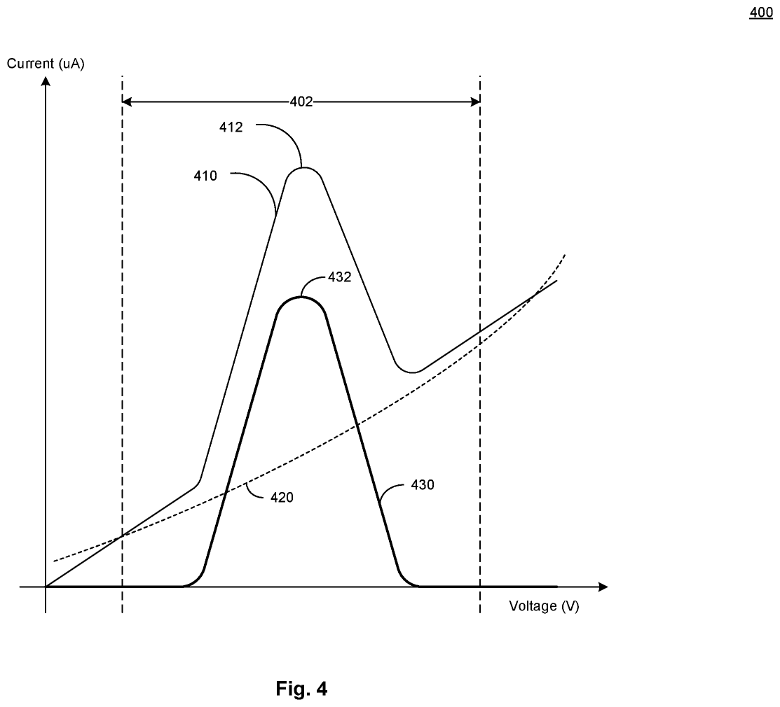

FIG. 4 illustrates an example biochemical sensor response, in accordance with present implementations. In some implementations, the example electronic sensor device 300 exhibits the example biochemical sensor response 400 in accordance with present implementations. As illustrated by way of example in FIG. 4, the example biochemical sensor response is bounded by a characteristic voltage window 402, and includes a characteristic response current curve 410, a characteristic current peak 412, a baseline calibration curve 420, and corrected characteristic response current 430, and a corrected current peak 432. In some implementations, voltammetry-based approaches uniquely leverage the electroactive nature of target drug molecules for quantification, thus eliminating reliance on the availability of recognition elements, mediators, and the like. In some implementations, pulse voltammetry, including but not limited to differential pulse voltammetry (DPV) and square wave voltammetry (SWV) are advantageous for the quantification of electroactive species due to their ability to suppress non-Faradaic background current. In some implementations, an example system sweeps voltage across the biochemical sensor electrode 210 and the reference electrode 230 above redox potential of target electroactive species. As one example, a redox potential is an oxidation potential. In some implementations, a characteristic current peak 412 is recorded at a fingerprint redox voltage associated with a target biochemical, with a peak height correlated to a concentration level of the target biochemical.

The characteristic voltage window 402 includes and bounds a range of voltages associated with the characteristic current peak 412 of the characteristic response current curve 410. In some implementations, the characteristic voltage window 402 includes a predetermined maximum window voltage and a predetermined minimum window voltage. In some implementations, the maximum window voltage and the minimum window voltage of the characteristic voltage window 402 are predetermined to enclose, encompass, bound, or the like, a voltammogram defining an electrochemical response of a biochemical.

The characteristic response current curve 410 defines an electrochemical response of a particular biochemical, and includes a characteristic current peak 412 defining a maximum electrochemical response to voltage stimulation by the iontophoresis inducer 340. In some implementations, the characteristic response current curve 410 is associated with a particular biochemical. In some implementations, the characteristic response current curve 410 includes an electrochemical response from at least one of a target biochemical, background electrochemical activity, and an interferent present with the target biochemical. In some implementations, the characteristic current peak 412 of the characteristic response current curve 410 has a particular current magnitude associated with a concentration of the target biochemical. Thus, in some implementations, the example device 100 determines a concentration of target biochemical present in the biofluid of the biological surface 380 based on a magnitude of a current peak. However, in some implementations, the characteristic current peak 412 is distorted by the presence of background electrochemical activity and an interferent present with the target biochemical. Thus, in some implementations, mitigation of one or more of these distortion drivers is conducted.

The baseline calibration curve 420 defines a level of background electrochemical activity present in the characteristic voltage window 402. In some implementations, the baseline calibration curve 420 is physically detected by obtaining current responses from a biofluid not including a target biochemical or an interferent. In some implementations, the baseline calibration curve 420 is generated based on a curve fitted to a physically detected calibration current response, or a predetermined value based on an estimate thereon. In some implementations, baseline calibration curve 420 is or is based on a combination of a 3rd-order polynomial and exponential equation. In some implementations, the polynomial and exponential equation includes various constants associated with background electrical activity present within the characteristic voltage window 402. In some implementations, Equation 1 (Eq. 1) corresponds to a baseline calibration curve in accordance with present implementations. It is to be understood that the baseline calibration curve 420 is variable with respect to voltage windows and target biochemicals. Thus, in some implementations, the baseline calibration curve 420 is different with respect to varying voltage windows and target biochemicals.

I_{baseline}=a₁V³+a₂V+a₃+a₄×e^{a}^{₅}^{V} Eq. 1

The corrected characteristic response current 430 defines a characteristic response current associated with a target biochemical and excluding background electrochemical activity. In some implementations, the corrected characteristic response current 430 is generated by subtracting a baseline calibration current value at a particular voltage value from a characteristic response current value at the particular voltage value, for all or a substantial portion of the voltage values within the voltage window. In some implementations, Equation 2 (Eq. 2) corresponds to a corrected characteristic response current curve 430 in accordance with present implementations.

Iₓ^{corrected}=Iₓ^{characteristic}−Iₓ^{baseline}, Vₘᵢₙ≤x≤Vₘₐₓ Eq. 2

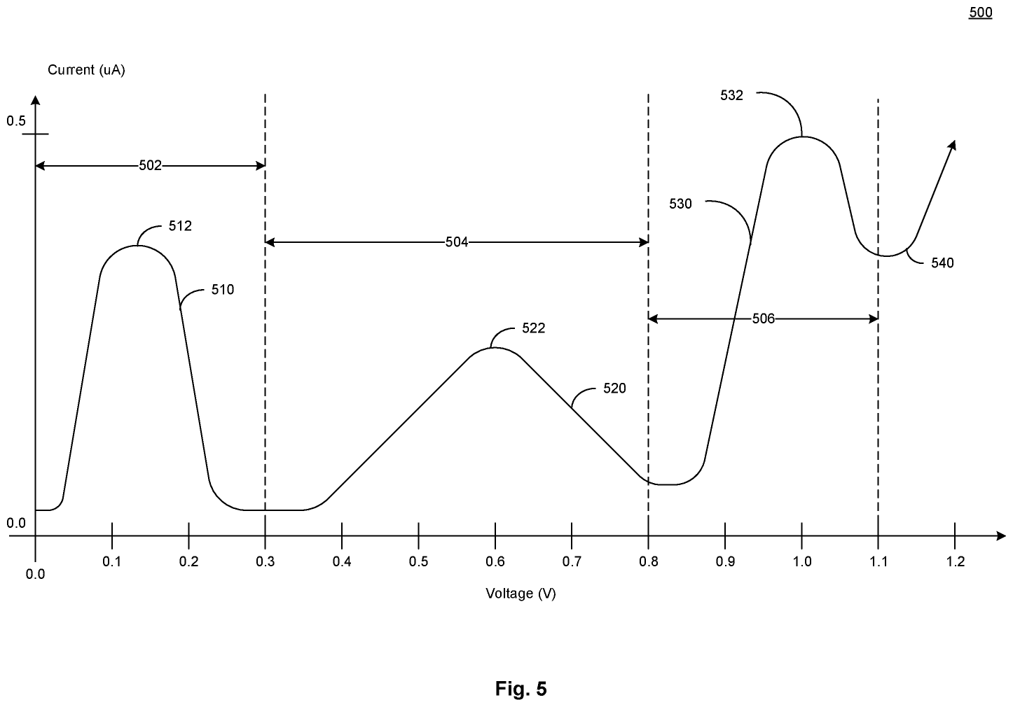

FIG. 5 illustrates an example biochemical sensor response including multiple biochemical sensor windows, in accordance with present implementations. In some implementations, the example electronic sensor device 300 exhibits the example biochemical sensor response 500 including one or more of a first characteristic voltage window 502, a second characteristic voltage window 504 and a third characteristic voltage window 506 in accordance with present implementations. The example biochemical sensor response 500 includes a first characteristic response current curve 510 associated with a first biochemical, a second characteristic response current curve 520 associated with a second biochemical, a third characteristic response current curve 530 associated with a third biochemical, and a fourth characteristic response current 532 associated with the third biochemical.

In some implementations, voltammetric quantification supports at least three biochemicals, including dipyridamole (DP), acetaminophen (APAP), and caffeine (CAFF). In some implementations, DP is electrochemically oxidized at a relatively low voltage potential of less than 0.5 V. In some implementations, APAP is electrochemically oxidized at a relatively moderate voltage potential between 0.5 V and 1.0 V. In some implementations, CAFF is electrochemically oxidized at a relatively high voltage potential of 0.8 V and above. It is advantageous to detect one or more of these biochemical due to their significance in disease treatment and necessity for therapeutic monitoring in biological fluids. For example, DP is used for cardiovascular disease treatment due to its vasodilating and antiplatelet properties. As one example, monitoring of antiplatelet therapies are advantageous to stroke patients. As another example, APAP is a widely-used pain reliever and fever reducer with remarkable variation in metabolism, and therapeutic monitoring is advantageous to control patient exposure to potentially toxic effects thereof. As another example, CAFF is advantageous to treatment of airway obstruction and prematurity apnea, and therapeutic monitoring is advantageous in individualizing dosage to reduce drug toxicity.

The first characteristic voltage window 502 includes and bounds a range of voltages associated with the first characteristic current peak 512 of the first characteristic response current curve 510. In some implementations, the first characteristic voltage window 502 includes a predetermined maximum window voltage and a predetermined minimum window voltage. In some implementations, the maximum window voltage and the minimum window voltage of the second characteristic voltage window 502 are predetermined to enclose, encompass, bound, or the like, a voltammogram defining an electrochemical response of DP. In some implementations, the minimum voltage and the maximum window voltage of the first characteristic voltage window 502 are respectively 0.0 V and 0.3 V and are associated with electrochemical responses of DP. In some implementations, the first characteristic current peak 512 indicates presence of DP in concentrations ranging between 0.05 uM to 10 uM. In some implementations, the concentrations are nonlinearly correlated to a current response range from 0.0 uA to 0.3 uA. In some implementations, the first characteristic response current curve 510 is responsive to DP in the absence of any interferent.

The second characteristic voltage window 504 includes and bounds a range of voltages associated with the second characteristic current peak 522 of the second characteristic response current curve 520. In some implementations, the second characteristic voltage window 504 includes a predetermined maximum window voltage and a predetermined minimum window voltage. In some implementations, the maximum window voltage and the minimum window voltage of the second characteristic voltage window 504 are predetermined to enclose, encompass, bound, or the like, a voltammogram defining an electrochemical response of APAP. In some implementations, the minimum voltage and the maximum window voltage of the second characteristic voltage window 504 are respectively 0.3 V and 0.8 V and are associated with electrochemical responses of APAP. In some implementations, the second characteristic current peak 522 indicates presence of APAP in concentrations ranging between 0.05 uM to 10 uM. In some implementations, the concentrations are linearly correlated to a current response range from 0.0 uA to 0.12 uA. In some implementations, the second characteristic response current curve 520 is responsive to APAP in the absence of any interferent.

The third characteristic voltage window 506 includes and bounds a range of voltages associated with the third characteristic current peak 532 of the third characteristic response current curve 530. In some implementations, the third characteristic voltage window 506 includes a predetermined maximum window voltage and a predetermined minimum window voltage. In some implementations, the maximum window voltage and the minimum window voltage of the third characteristic voltage window 506 are predetermined to enclose, encompass, bound, or the like, a voltammogram defining an electrochemical response of CAFF. In some implementations, the minimum voltage and the maximum window voltage of the second characteristic voltage window 504 are respectively 0.8 V and 1.1 V and are associated with electrochemical responses of CAFF. In some implementations, the third characteristic current peak 532 indicates presence of CAFF in concentrations ranging between 0.05 uM to 10 uM. In some implementations, the concentrations are linearly correlated to a current response range from 0.0 uA to 0.6 uA. In some implementations, the third characteristic response current curve 530 is responsive to CAFF in the absence of any interferent. The fourth characteristic response current curve 540 is outside any voltage window associated with DP, APA, and CAFF. In some implementations, the fourth characteristic response current curve is monotonically increasing within a maximum operating sensor range of the biochemical sensor electrode 210.

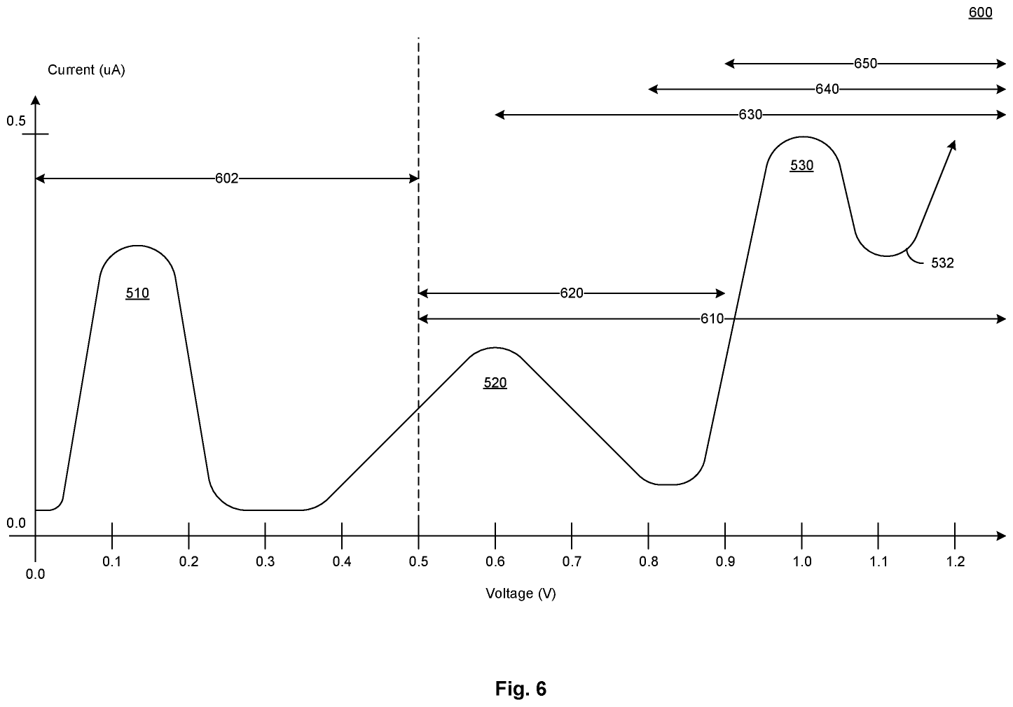

FIG. 6 illustrates an example biochemical sensor response including multiple interferent windows, in accordance with present implementations. In some implementations, the example electronic sensor device 300 exhibits the example biochemical sensor response 600 including one or more of the first characteristic response current curve 510, the characteristic response current 504, the third characteristic characteristic response current 506, the fourth characteristic response current 532, noninterference voltage window 602, a first interferent voltage window 610, a second interferent voltage window 620, a third interferent voltage window 630, a fourth interferent voltage window 640, and a fifth interferent voltage window 650, in accordance with present implementations.

The noninterference voltage window 602 includes and bounds a range of voltages outside the distortion effects of one or more interferents. In some implementations, the noninterference voltage window 602 includes a predetermined maximum window voltage and a predetermined minimum window voltage. In some implementations, the maximum window voltage and the minimum window voltage of the noninterference voltage window 602 are predetermined to enclose, encompass, bound, or the like, a voltammogram defining an electrochemical response outside the distortion effects of one or more interferents. In some implementations, the minimum voltage and the maximum window voltage of the noninterference voltage window 602 are respectively 0.0 V and 0.5 V.

The first interferent voltage window 610 includes and bounds a range of voltages associated with the interferent biochemical tryptophan (TRY). In some implementations, the first interferent voltage window 610 includes a predetermined maximum window voltage and a predetermined minimum window voltage. In some implementations, the maximum window voltage and the minimum window voltage of the first interferent voltage window 610 are predetermined to enclose, encompass, bound, or the like, a voltammogram defining an electrochemical response of TRY. In some implementations, the minimum voltage and the maximum window voltage of the first interferent voltage window 610 are respectively 0.5 V and 1.2 V and are associated with electrochemical responses of TRY. In some implementations, a first interferent current peak indicates presence of TRY. In some implementations, the current response associated with TRY ranges from 0.0 uA to 1.5 uA in concentrations ranging between 5 uM to 34 uM, in the absence of any biochemical and other interferent.

The second interferent voltage window 620 includes and bounds a range of voltages associated with the interferent biochemical uric acid (UA). In some implementations, the second interferent voltage window 620 includes a predetermined maximum window voltage and a predetermined minimum window voltage. In some implementations, the maximum window voltage and the minimum window voltage of the second interferent voltage window 620 are predetermined to enclose, encompass, bound, or the like, a voltammogram defining an electrochemical response of UA. In some implementations, the minimum voltage and the maximum window voltage of the second interferent voltage window 620 are respectively 0.5 V and 0.9 V and are associated with electrochemical responses of UA. In some implementations, a second interferent current peak indicates presence of UA. In some implementations, the current response associated with UA ranges from 0.0 uA to 0.2 uA in concentrations ranging between 18 uM to 32 uM, in the absence of any biochemical and other interferent.

The third interferent voltage window 630 includes and bounds a range of voltages associated with the interferent biochemical tyrosine (TYR). In some implementations, the third interferent voltage window 630 includes a predetermined maximum window voltage and a predetermined minimum window voltage. In some implementations, the maximum window voltage and the minimum window voltage of the third interferent voltage window 630 are predetermined to enclose, encompass, bound, or the like, a voltammogram defining an electrochemical response of TYR. In some implementations, the minimum voltage and the maximum window voltage of the third interferent voltage window 630 are respectively 0.6 V and 1.2 V and are associated with electrochemical responses of TYR. In some implementations, a third interferent current peak indicates presence of TYR. In some implementations, the current response associated with TYR ranges from 0.0 uA to 0.3 uA in concentrations ranging between 5 uM to 39 uM, in the absence of any biochemical and other interferent.

The fourth interferent voltage window 640 includes and bounds a range of voltages associated with the interferent biochemical histidine (HIS). In some implementations, the fourth interferent voltage window 640 includes a predetermined maximum window voltage and a predetermined minimum window voltage. In some implementations, the maximum window voltage and the minimum window voltage of the fourth interferent voltage window 640 are predetermined to enclose, encompass, bound, or the like, a voltammogram defining an electrochemical response of HIS. In some implementations, the minimum voltage and the maximum window voltage of the fourth interferent voltage window 640 are respectively 0.8 V and 1.2 V and are associated with electrochemical responses of HIS. In some implementations, a fourth interferent current peak indicates presence of HIS. In some implementations, the current response associated with HIS ranges from 0.0 uA to 2.0 uA in concentrations ranging between 258 uM to 742 uM, in the absence of any biochemical and other interferent.

The fifth interferent voltage window 650 includes and bounds a range of voltages associated with the interferent biochemical methionine (MET). In some implementations, the fourth interferent voltage window 650 includes a predetermined maximum window voltage and a predetermined minimum window voltage. In some implementations, the maximum window voltage and the minimum window voltage of the fifth interferent voltage window 650 are predetermined to enclose, encompass, bound, or the like, a voltammogram defining an electrochemical response of MET. In some implementations, the minimum voltage and the maximum window voltage of the fifth interferent voltage window 650 are respectively 0.9 V and 1.2 V and are associated with electrochemical responses of MET. In some implementations, a fifth interferent current peak indicates presence of MET. In some implementations, the current response associated with MET ranges from 0.0 uA to 1.5 uA in concentrations ranging between 3 uM to 17 uM, in the absence of any biochemical and other interferent.

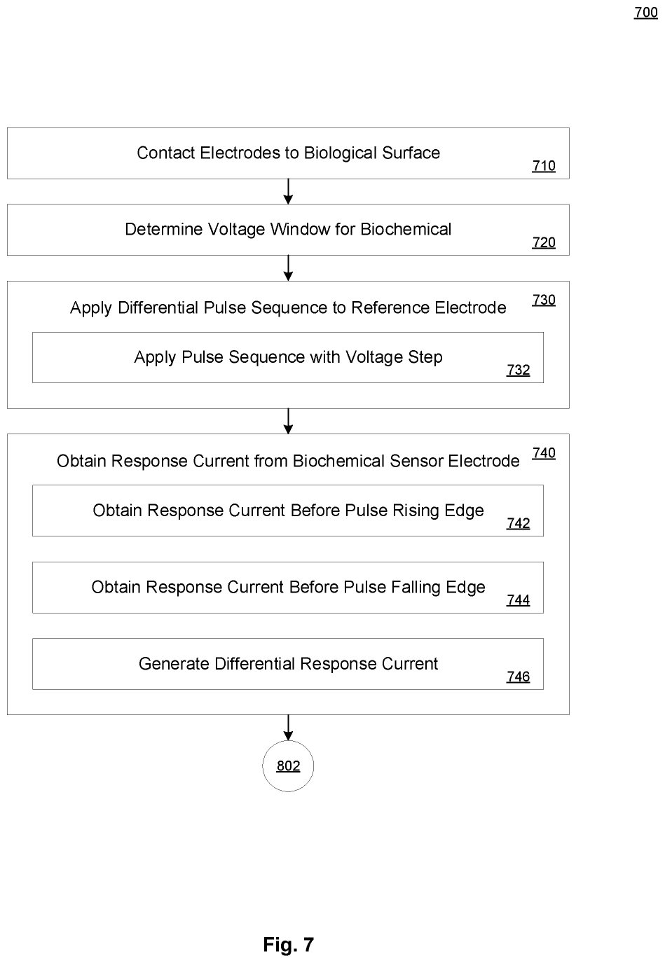

FIG. 7 illustrates an example method of electrically sensing a biochemical in accordance with present implementations. In some implementations, the example device 100 performs method 700 according to present implementations. In some implementations, the method 700 begins at step 710.

At step 710, the example system contacts electrodes to a biological surface. In some implementations, the biochemical sensor device 100 is a wearable device attached, affixed, or the like to a biological surface of an individual user's body. In some implementations, the biochemical sensor device is attached to a limb, arm, forearm, hand, or the like. In some implementations, the biochemical sensor surface 110 is disposed in contact with the biological surface 350, such that one or more of the biochemical sensor electrode 210, the counter electrode 220, and the reference electrode 230 are in contact with the biological surface 350. The method 700 then continues to step 720.

At step 720, the example system determines a voltage window for a biochemical. In some implementations, the system processor 310 determines a voltage window based on a selection, identification, input, or the like, designating a target biochemical. In some implementations, the target biochemical is one of DP, APA, and CAFF. The method 700 then continues to step 730.

At step 730, the example system applies a differential pulse sequence to a reference electrode. In some implementations, system processor 310 determines one or more parameters governing the electrical characteristics of the differential pulse sequence. In some implementations, the system processor 310 determines at least one of a pulse amplitude, a pulse period between pulses, a pulse width of each pulse, and a step magnitude of the differential pulse sequence. In some implementations, a pulse amplitude is between 0.0 V and 0.2 V. In some implementations, a pulse period is greater than 0.5 s. In some implementations, a pulse width is less than 0.2 s. In some implementations, step 730 includes step 732. At step 732, the example system applies a pulse sequence with a voltage step. In some implementations, the voltage step is monotonically increasing. In some implementations, the voltage step causes the differential pulse sequence to monotonically increase from a minimum voltage associated with a voltage window to a maximum voltage associated with the voltage window. In some implementations, the voltage step is added to a falling edge of the pulse. Thus, in some implementations, the voltage pulse ends at an ending voltage after the pulse that is higher than a starting voltage before the pulse, by an amount of the voltage step. The method 700 then continues to step 740.

At step 740, the example system obtains a response current from a biochemical sensor electrode. In some implementations, the system processor 310 obtains the response current from one or more of the TIA 350 and the ADC 360. In some implementations, the example system obtains the response current as an analog signal responsive to physical biochemical input, and generates a digital instruction, value, or the like, based on the analog signal. In some implementations, step 740 includes at least one of steps 742, 744 and 746. At step 742, the example system obtains a response current before a pulse rising edge. In some implementations, at least one of the system processor 310, the TIA 350 and the ADC 360 detects and captures a rising edge sample prior to the occurrence of a rising edge pulse. At step 744, the example system obtains a response current after a pulse falling edge. In some implementations, at least one of the system processor 310, the TIA 350 and the ADC 360 detects and captures a rising edge sample after the occurrence of a falling edge pulse. At step 746, the example system generates a differential response current. In some implementations, the system processor 310 generates the differential response current by averaging or the like two adjacent rising edge samples. In some implementations, the system processor 310 generates the differential response current by averaging or the like two adjacent falling edge samples. In some implementations, the method 700 then continues to step 802.

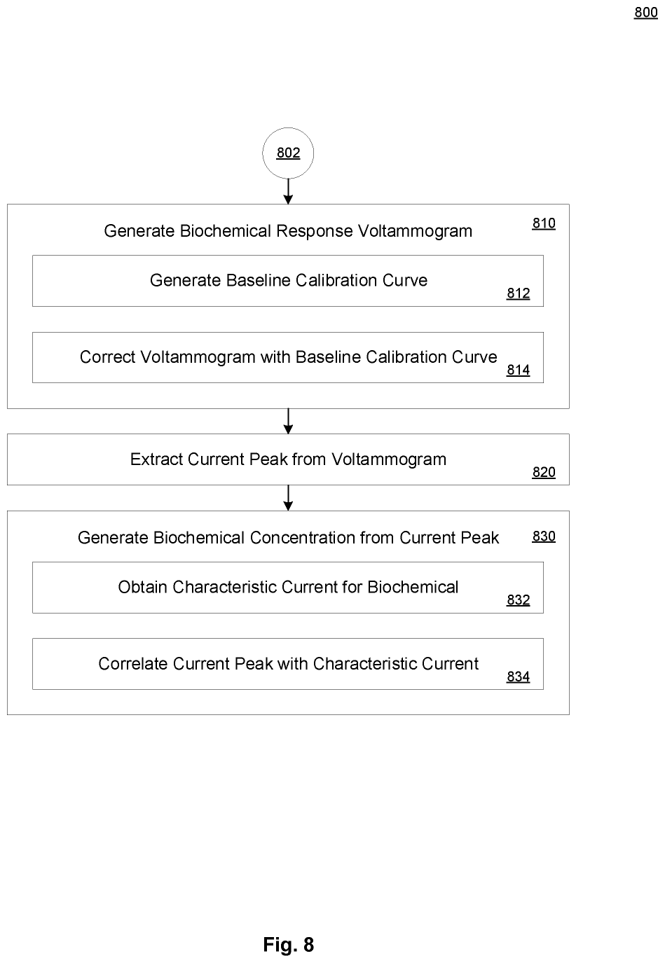

FIG. 8 illustrates an example method of electrically sensing a biochemical, further to the example method of FIG. 7. In some implementations, the example device 100 performs method 800 according to present implementations. In some implementations, the method 800 begins at step 802. The method 800 then continues to step 810.

At step 810, the example system generates a biochemical response voltammogram. In some implementations, the system processor 310 generates the biochemical response voltammogram by obtaining voltage and current response pairs. In some implementations, the biochemical response voltammogram includes a plurality of voltage and current response pairs respectively based on the differential response currents and the voltage magnitudes monotonically creasing by voltage step. In some implementations, step 810 includes at least one of steps 812 and 814. At step 812, the example system generates a baseline calibration curve. In some implementations, the system processor 310 generates, obtains, or the like, the baseline calibration curve in accordance with Eq. 1. At step 814, the example system corrects a votlammogram based at least partially on the baseline calibration curve. In some implementations, the system processor 310 corrects the baseline calibration curve in accordance with Eq. 2. The method 800 then continues to step 820.

At step 820, the example system extracts a current peak from the voltammogram. In some implementations, the system processor extracts the current peak by derivative. Alternatively, in some implementations, the system processor 310 transmits the voltammogram to an external processor, remote device, or the like, by the communication interface 370. In some implementations, the example system extracts a current peak for DP in the presence of one or more of UA, TRY, TYR, HIS and MET. In some implementations, the example system extracts a current peak for APAP in the presence of one or more of HIS and MET. In some implementations, the example system extracts a current peak for CAFF in the absence of UA, TRY, TYR, HIS and MET. The method 800 then continues to step 830.

At step 830, the example system generates a biochemical concentration from the current peak. In some implementations, the system processor 310 generates the biochemical concentration. In some implementations, step 830 includes at least one of steps 832 and 834. At step 832, the example system obtains a characteristic current for a biochemical. In some implementations, the system processor obtains, generates, or the like, a predetermined relationship between response current an concentration associated with a particular biochemical. As one example, the system processor 310 can retrieve a correlation between biochemical concentrations and current response magnitudes for one of DP, APAP, and CAFF. In some implementations, the correlation defines one or more linear or nonlinear relationships between response current magnitude and concentration of a particular biochemical. At step 834, the example system correlates the current peak with the characteristic current. In some implementations, the system processor 310 generates the biochemical concentration based on the magnitude of the extracted peak with respect to a linear, nonlinear, or like function correlating a biochemical with a ranges of concentrations based on magnitude of response current. In some implementations, the method 800 ends at step 830.

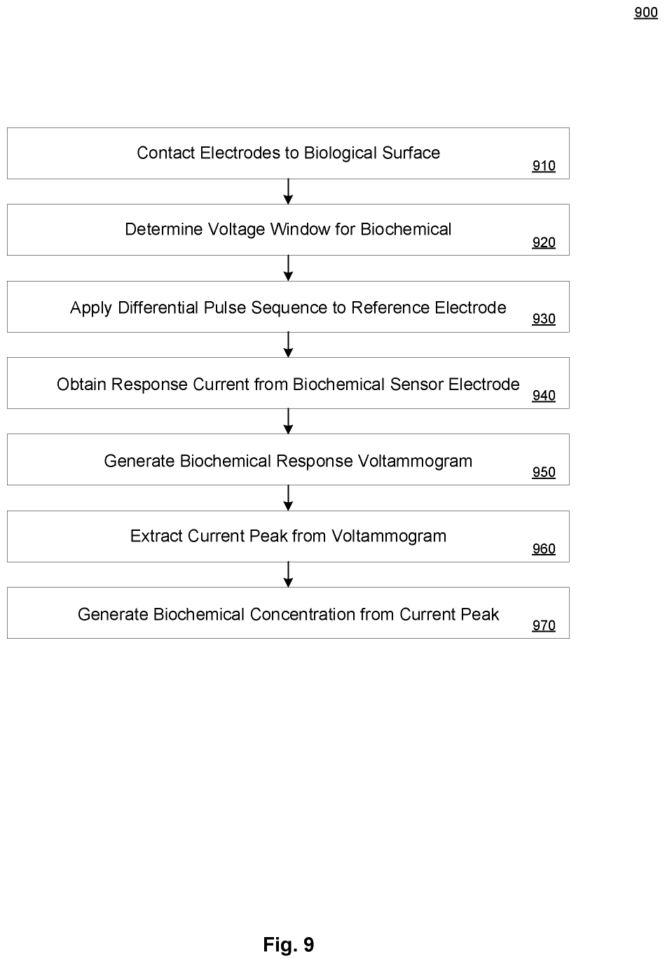

FIG. 9 illustrates a further example method of electrically sensing a biochemical. In some implementations, the example device 100 performs method 900 according to present implementations. In some implementations, the method 900 begins at step 910.

At step 910, the example system contacts electrodes to a biological surface. In some implementations, step 910 corresponds to step 710. The method 900 then continues to step 920. At step 920, the example system determines a voltage window for a biochemical. In some implementations, step 920 corresponds to step 720. The method 900 then continues to step 930. At step 930, the example system applies a differential pulse sequence to a reference electrode. In some implementations, step 930 corresponds to step 730. The method 900 then continues to step 940. At step 940, the example system obtains a response current from a biochemical sensor electrode. In some implementations, step 940 corresponds to step 740. In some implementations, the method 900 then continues to step 950. At step 950, the example system generates a biochemical response voltammogram. In some implementations, step 950 corresponds to step 810. The method 900 then continues to step 960. At step 960, the example system extracts a current peak from the voltammogram. In some implementations, step 960 corresponds to step 820. The method 900 then continues to step 970. At step 970, the example system generates a biochemical concentration from the current peak. In some implementations, step 970 corresponds to step 830. In some implementations, the method 900 ends at step 970.

The herein described subject matter sometimes illustrates different components contained within, or connected with, different other components. It is to be understood that such depicted architectures are illustrative, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected,” or “operably coupled,” to each other to achieve the desired functionality, and any two components capable of being so associated can also be viewed as being “operably couplable,” to each other to achieve the desired functionality. Specific examples of operably couplable include but are not limited to physically mateable and/or physically interacting components and/or wirelessly interactable and/or wirelessly interacting components and/or logically interacting and/or logically interactable components

With respect to the use of plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.).

Although the figures and description may illustrate a specific order of method steps, the order of such steps may differ from what is depicted and described, unless specified differently above. Also, two or more steps may be performed concurrently or with partial concurrence, unless specified differently above. Such variation may depend, for example, on the software and hardware systems chosen and on designer choice. All such variations are within the scope of the disclosure. Likewise, software implementations of the described methods could be accomplished with standard programming techniques with rule-based logic and other logic to accomplish the various connection steps, processing steps, comparison steps, and decision steps.

It will be further understood by those within the art that if a specific number of an introduced claim recitation is intended, such an intent will be explicitly recited in the claim, and in the absence of such recitation, no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations).

Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general, such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

Further, unless otherwise noted, the use of the words “approximate,” “about,” “around,” “substantially,” etc., mean plus or minus ten percent.

The foregoing description of illustrative implementations has been presented for purposes of illustration and of description. It is not intended to be exhaustive or limiting with respect to the precise form disclosed, and modifications and variations are possible in light of the above teachings or may be acquired from practice of the disclosed implementations. It is intended that the scope of the invention be defined by the claims appended hereto and their equivalents.

1. A method of noninvasively detecting a biochemical in a biofluid, comprising:
applying a voltage pulse having a magnitude within a biochemical voltage window associated a biochemical;
obtaining a response current from a biochemical sensor electrode;
generating a biochemical response voltammogram based on the response current;
extracting a current peak from the biochemical response voltammogram; and
generating a biochemical concentration based on the current peak.

2. The method of claim 1, wherein the applying the voltage pulse further comprises applying a differential pulse sequence including the voltage pulse to the reference electrode.

3. The method of claim 2, wherein the applying the differential pulse sequence further comprises applying the differential pulse sequence to the reference electrode at an increasing voltage step.

4. The method of claim 1, further comprising:
obtaining the current response before an edge of the voltage pulse.

5. The method of claim 4, wherein the edge is a rising edge.

6. The method of claim 4, wherein the edge is a falling edge.

7. The method of claim 1, wherein the voltage pulse is a square wave.

8. The method of claim 1, further comprising:
generating a differential current response based on the response current,
wherein the biochemical response voltammogram is based on the differential current response.

9. The method of claim 8, wherein the differential current response is based on a difference between a plurality of current values obtained at times separated by a predetermined time interval.

10. The method of claim 1, further comprising:
correcting the biochemical response voltammogram with a baseline calibration curve.

11. The method of claim 10, wherein the correcting the biochemical response voltammogram comprises shifting the biochemical response voltammogram by a baseline current magnitude value of the baseline calibration curve.

12. The method of claim 10, wherein the correcting the biochemical response voltammogram comprises subtracting a baseline current magnitude value of the baseline calibration curve associated with a particular voltage value from a corresponding response current magnitude value of the biochemical response voltammogram associated with the particular voltage.

13. The method of claim 10, further comprising:
generating the baseline calibration curve based at least in part on a polynomial equation.

14. The method of claim 1, further comprising contacting the biochemical sensor electrode and the reference electrode to a biological surface.

15. The method of claim 14, wherein the biological surface comprises human skin.

16. The method of claim 1, wherein the biochemical is obtained from human sweat.

17. The method of claim 1, wherein the biochemical is dipyridamole, and the biochemical voltage window ranges between 0.0 V and 0.3 V.

18. The method of claim 1, wherein the biochemical is acetaminophen, and the biochemical voltage window ranges between 0.3 V and 0.8 V.

19. The method of claim 1, wherein the biochemical is caffeine, and the biochemical voltage window ranges between 0.8 V and 1.1 V.

20. The method of claim 1, wherein the biochemical is caffeine, and the biochemical voltage window ranges between 0.8 V and 1.1 V.

21. The method of claim 1, wherein the biochemical voltage window is disposed at least partially outside an interferent voltage window.

22. The method of claim 21, wherein the interferent is tryptophan, and the interferent voltage window ranges from 0.5 V and above.

23. The method of claim 21, wherein the interferent is uric acid, and the interferent voltage window ranges between 0.5 V and 0.9 V.

24. The method of claim 21, wherein the interferent is tyrosine, and the interferent voltage window ranges from 0.6 V and above.

25. The method of claim 21, wherein the interferent is histidine, and the interferent voltage window ranges from 0.8 V and above.

26. The method of claim 21, wherein the interferent is methionine, and the interferent voltage window ranges from 0.9 V and above.

27. A device to noninvasively detecting a biochemical in a biofluid, the electronic device comprising:
an iontophoresis inducer configured to apply a voltage pulse to a biofluid including a biochemical, the voltage pulse having a magnitude within a biochemical voltage window associated a biochemical;
a biochemical sensor electrode operatively configured to obtain a response current from the biofluid;
a transimpedance amplifier operatively coupled to the biochemical sensor electrode, and configured to obtain the response current from the biochemical sensor electrode; and
a system processor operatively coupled to the iontophoresis inducer and the transimpedance amplifier, and configured to generate a biochemical response voltammogram based on the response current, extract a current peak from the biochemical response voltammogram, and generate a biochemical concentration based on the current peak.

28. The device of claim 27 , wherein the iontophoresis inducer is further configured to apply a differential pulse sequence including the voltage pulse to the reference electrode.

29. The device of claim 28, wherein the iontophoresis inducer is further configured to apply the differential pulse sequence to the reference electrode at an increasing voltage step.

30. The device of claim 27, wherein the transimpedance amplifier is further configured to obtain the current response before an edge of the voltage pulse.

31. The device of claim 30, wherein the edge is a rising edge.

32. The device of claim 30, wherein the edge is a falling edge.

33. The device of claim 27, wherein the voltage pulse is a square wave.

34. The device of claim 27, further comprising:
a reference electrode operatively coupled to the iontophoresis inducer, and configured to apply the voltage pulse to the biofluid.

35. The device of claim 27, wherein the biochemical sensor electrode comprises a boron-doped diamond electrode.

36. The device of claim 27, wherein a surface of the biochemical sensor electrode is hydrogen-terminated.

37. The device of claim 27, wherein a surface of the biochemical sensor electrode is oxygen-terminated.
